(19) United States
(12) Patent Application Publication
(10) Pub. No.: US 2021/0358612 A1
(43) Pub. Date: Nov. 18, 2021

(54) VALUE BASED HEALTH CARE CLAIMS PROCESSING SYSTEM

(71) Applicant: Contessa Health, LLC, Nashville, TN (US)

(72) Inventors: Travis James Messina, Nashville, TN (US); David Lee Coppeans, Nashville, TN (US)

(21) Appl. No.: 17/212,467

(22) Filed: Mar. 25, 2021

Related U.S. Application Data
(63) Continuation of application No. 15/043,155, filed on Feb. 12, 2016, now abandoned.
(60) Provisional application No. 62/232,165, filed on Sep. 24, 2015.

(51) Int. Cl. G16H 40/63; G06Q 10/10; G16Z 99/00

(52) U.S. Cl. CPC G16H 40/63; G16Z 99/00; G06Q 10/10

(57) ABSTRACT

A value based health care claims processing system for generating single health care claims for episodes of care includes databases for storing payor, provider, episode of care, procedure, provider episode claims, provider claims, and payor remittance claims data; a system management process and user interface for defining payor, provider, episode, and procedure information in the system; an import process and user interface for importing provider claims into the system; an episode management process and user interface for assigning provider claims data to episodes of care; and an export process and user interface for reconfiguring and exporting to payors single claims for episodes of care.

### CROSS-REFERENCES TO RELATED APPLICATIONS

The present application is a continuation of and claims the benefit to U.S. application Ser. No. 15/043,155, filed on Feb. 12, 2016 titled “Value Based Health Care Claims Processing System,” which is a non-provisional of U.S. Provisional Patent Application Ser. No. 62/232,165, filed on Sep. 24, 2015 titled “System and Method of Providing Value Based Health Care Services,” each of which is incorporated by reference in their entireties.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND OF THE INVENTION

The present disclosure relates generally to systems for processing health care claims.

More particularly, the present disclosure relates to a system for processing value based health care claims.

Health care claims processing systems are well known in the art. In a typical scenario, health care providers treat a patient and then submit individual claims for the services they have provided to an insurance company or entity responsible for processing payments known as a payor, which then processes the claims and remits payments to the providers. Prior art health care claims processing systems, however, are not designed to process value based health care claims, which have recently become more popular in the health care industry as a way to reduce costs.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present invention is directed to a value based health care claims processing system. In one embodiment, the system includes a payor, provider, episode, procedure data database for storing payor, provider, episode, and procedure data; a provider episode claims data database for storing provider episode claims data; and a provider claims data database for storing provider claims data. The system further includes a system management process and system management user interface in communication with the payor, provider, episode, and procedure data database, the provider episode claims data database, and the provider claims data database, that enables a user to define payor, provider, episode, and procedure information in the system; an import process and import user interface in communication with the provider claims data database that enables provider claims to be imported into the system; an episode management process and episode management user interface in communication with the provider episode claims data database for assigning provider claims data to episodes of care; and an export process and export user interface in communication with the provider episode claims data database that enables provider episode claims data to be reconfigured and exported to payors as single claims for episodes of care.

The system may also include a payer remittance claims data database for storing payer remittance claims data in communication with the import process and import user interface, the import process and import user interface operable to enable payor remittance data files to be imported into the system; a validation process and validation user interface in communication with the payor, provider, episode, and procedure data database, the provider claims data database, and the payor remittance claims data database and operable to validate provider claims data matches user defined parameters for information related to payor, provider, episode, and procedures; a reconciliation process and reconciliation user interface in communication with the provider claims data database and the payor remittance claims data database and operable to match provider claims data to remittance claims data; a reports and analytics data database for storing reports and analytics data in communication with the payor, provider, episode, and procedure data database, the provider episode claims data database, the provider claims data database, and the payor remittance claims data database; and a reporting and analytics process and reports/analytics user interface in communication with the reports and analytics data database and operable to compile data from claims, system, and remittance data to provide reporting and analytics.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a block diagram showing one embodiment of the value based health care claims processing system of the present invention.

FIG. 2 is a block diagram showing one embodiment of the claims system architecture of the present invention.

FIGS. 3-8 are screen shots generated by one embodiment of the present invention.

FIGS. 9A-9C show another illustration of the claims system architecture for one embodiment of the present invention.

FIGS. 10A-10B are a workflow diagram showing how claims data is processed through the claims system in one embodiment of the present invention.

FIG. 11 is a block diagram illustrating how an episode of care is constructed in one embodiment of the claims system of the present invention.

FIG. 12 is a block diagram showing how claim and system generated identifiers are linked to episode of care identifiers within the claims system of one embodiment of the present invention.

FIG. 13 is a block diagram illustrating how the episode of care identification system operates in one embodiment of the present invention.

FIGS. 14-29 are screen shots generated by a second embodiment of the present invention.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

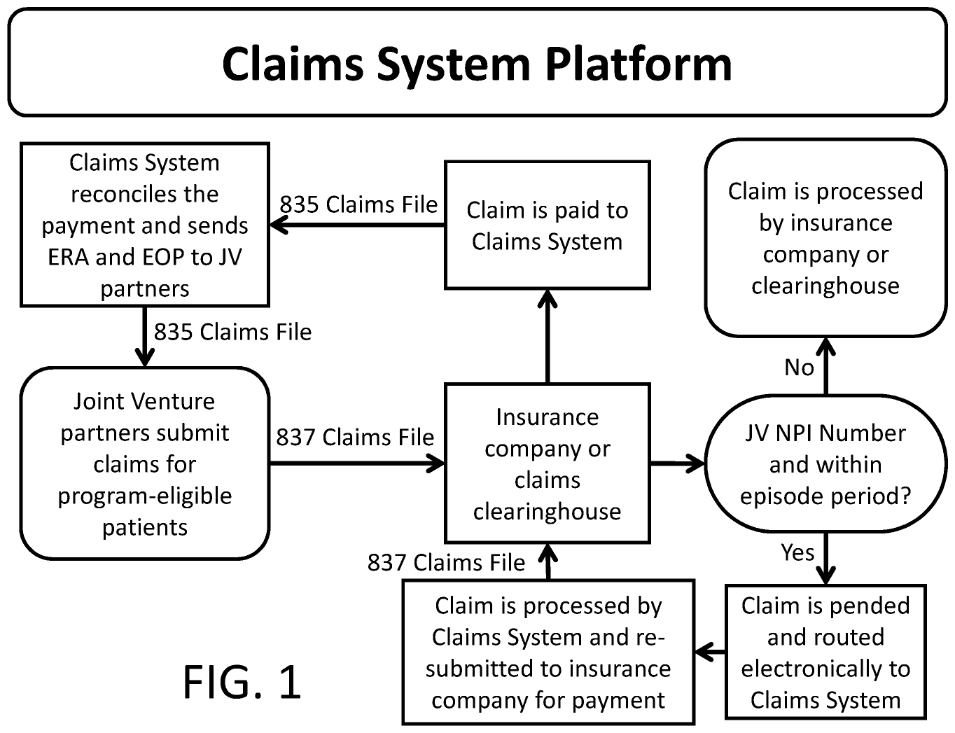

FIG. 1 shows an exemplary embodiment of the value based health care claims processing system of the present invention. Joint venture partners, which may include physicians, ambulances, ambulatory service centers, or any other type of healthcare provider, submit claims (in the form of 837 claim files in one embodiment) for program eligible patients to an insurance company or claims clearinghouse. The insurance company or claims clearinghouse checks to see if the claim files includes a joint venture national provider identifier number within an episode period and, if so, pends the claims and routes them electronically to the claims system 10 of the present invention. If not, the claims are processed by the insurance company or clearinghouse according to standard protocols.

The routed claims are then processed by the claims system 10 of the present invention and re-submitted (as a 837 claim file in one embodiment) to the insurance company or claims clearinghouse for payment. The insurance company or claims clearinghouse generates payment (via an 835 claim file in one embodiment), which is submitted to the claims system 10 of the present invention. The claims system 10 reconciles the payment and then sends payment (via 835 files) to the joint venture partners.

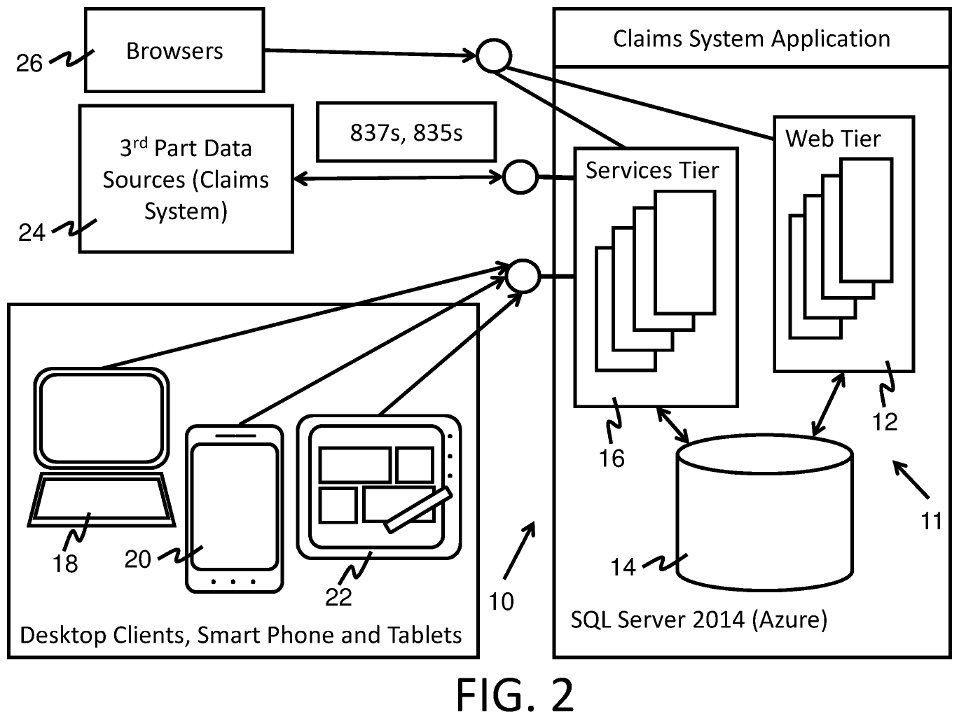

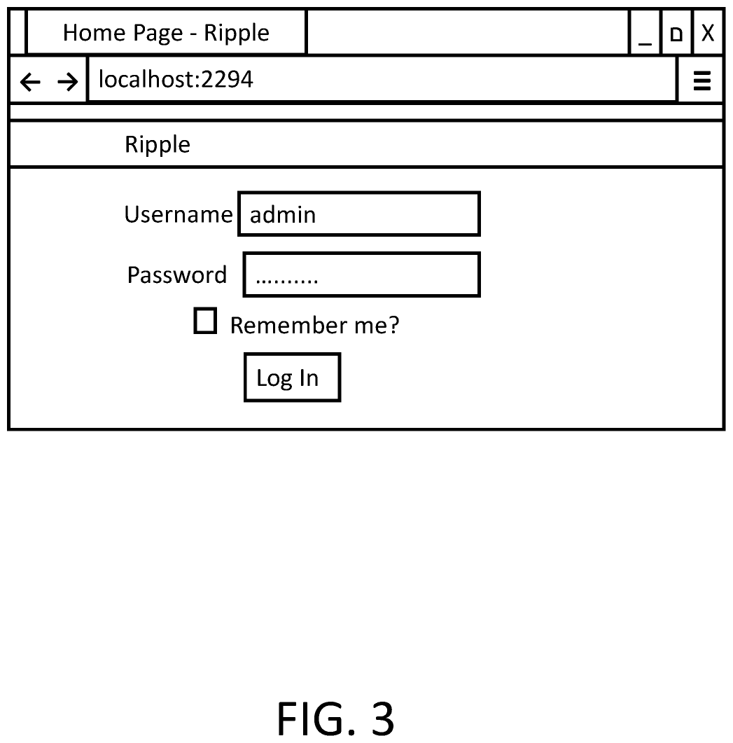

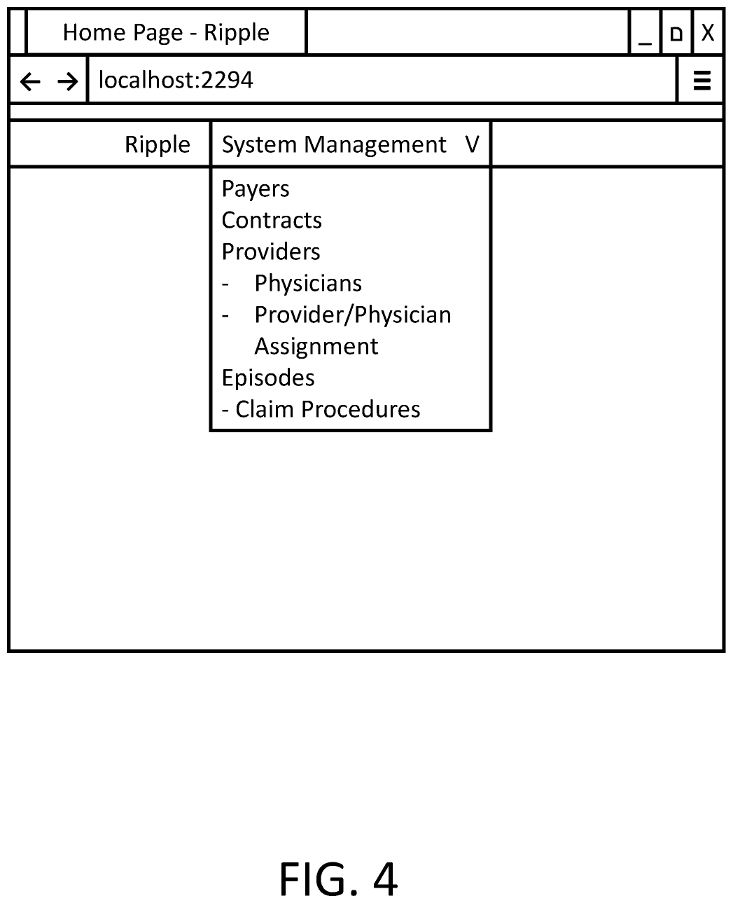

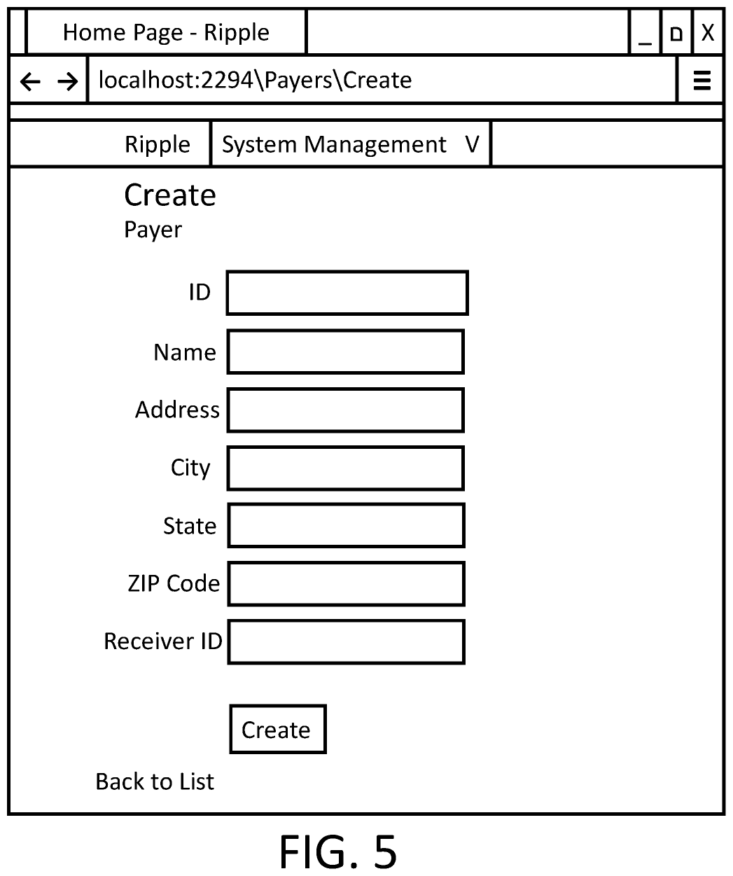

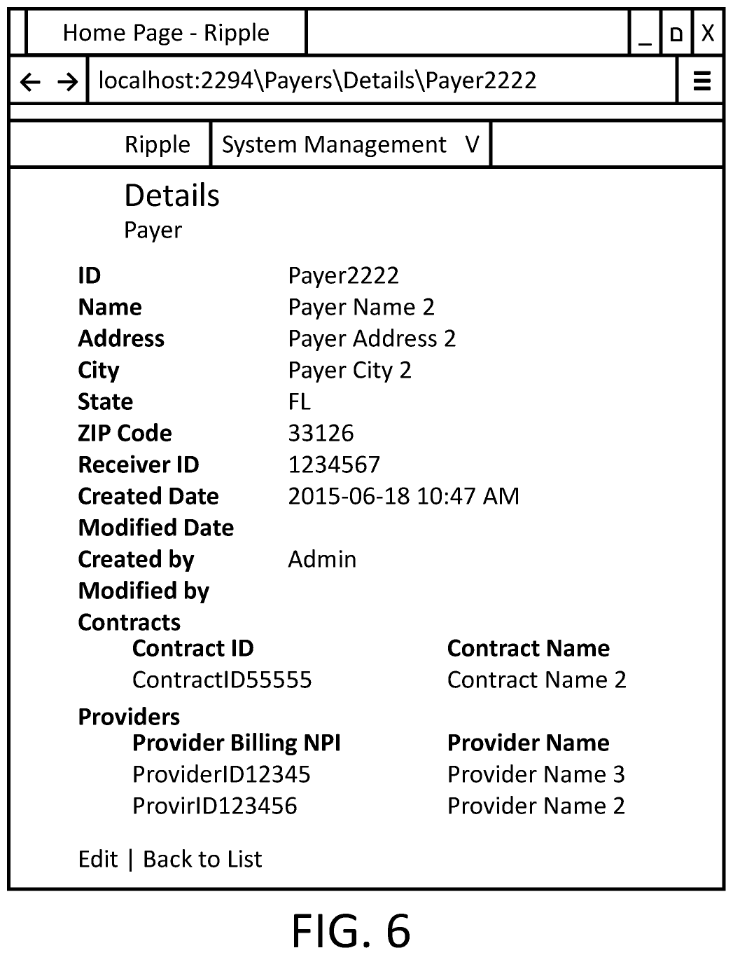

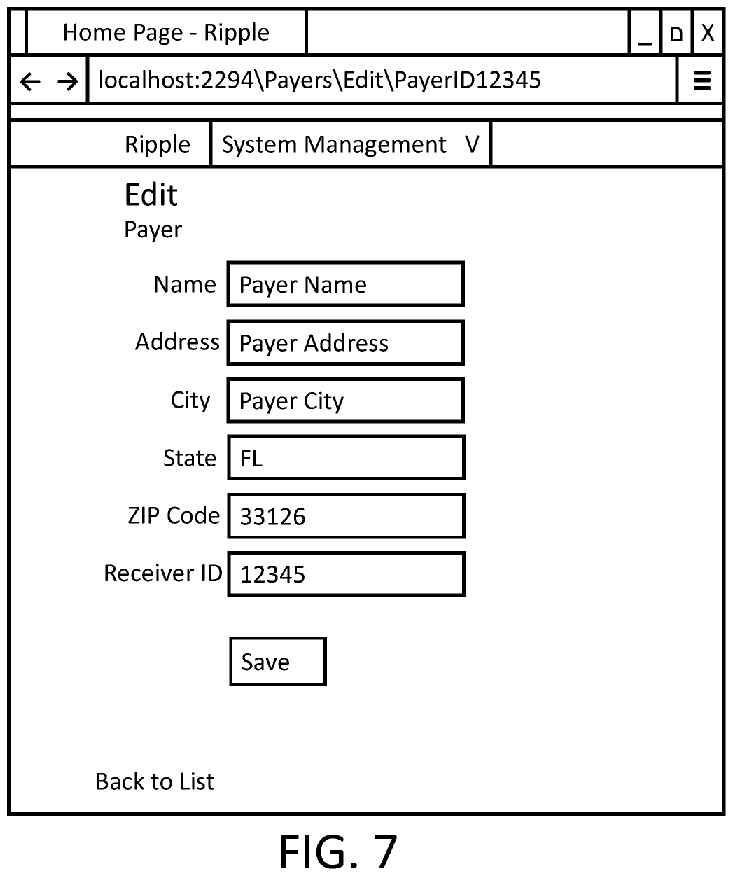

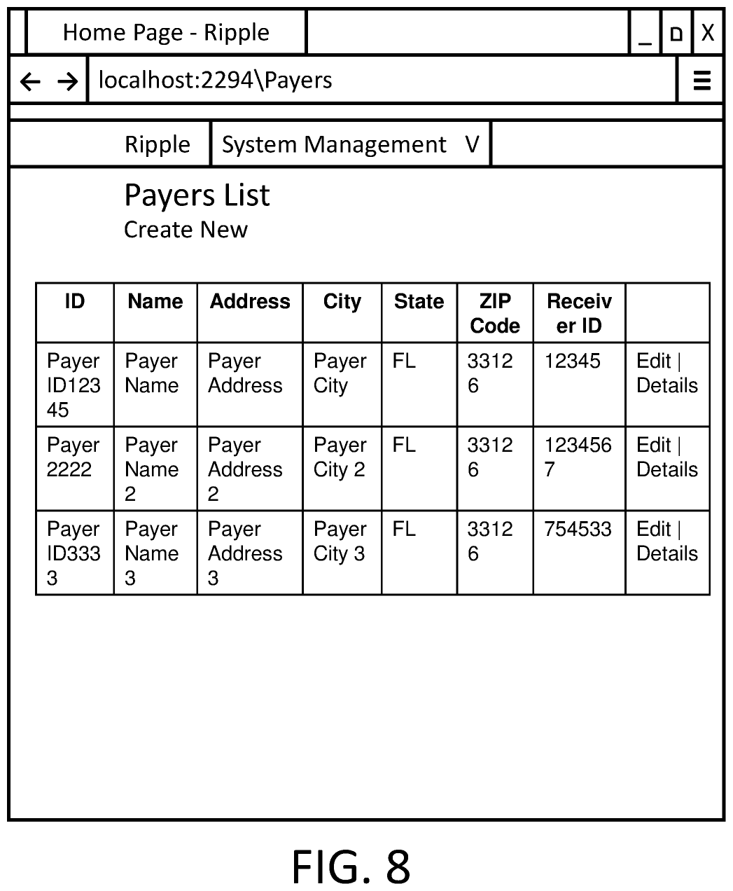

Referring to FIG. 2, one embodiment of the claims system 10 of the present invention includes a main application 11 that is hosted in the cloud on a Microsoft Azure platform. The system 10 includes a Web Tier 12, which uses the Azure Web App (not shown), an application database 14 hosted on Azure SOL, and a Services Tier 16, which is configured using Azure API (Application Program Interface) Management tools. Azure Web Apps may be deployed to particular geographical regions and scaled as needed. Azure SOL is HIPAA-compliant and secure. Microsoft executes Business Associate Agreements (BAAs) related to HIPAA best practices for these systems. The software code included in main application 11 may be developed using Microsoft Visual Studio.

Desktop clients 18, smart phones 20, and tablets 22 can connect to the claims system 10 using the Services Tier 16. Third party data sources 24, e.g., third party claims systems, can likewise connect to and submit claims (837 transaction files) and receive payments (835 transaction files) for those claims using the Services Tier 16. Browsers 26 running on conventional computers can be used to connect to the claims system 10 using both the Web Tier 12 and the Services Tier 16.

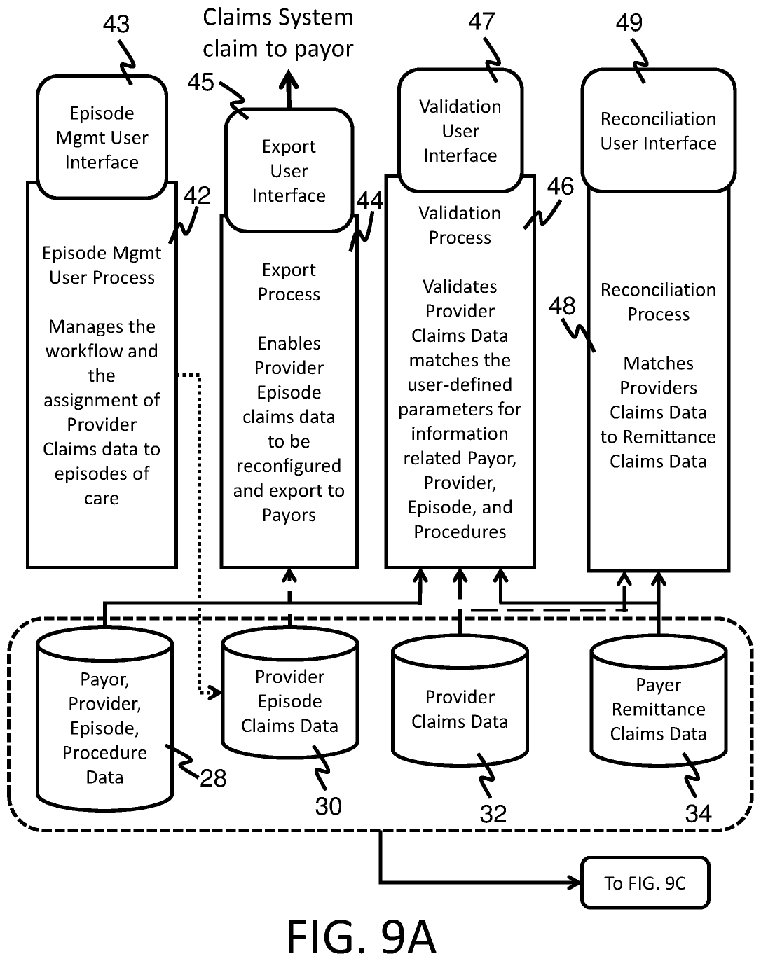

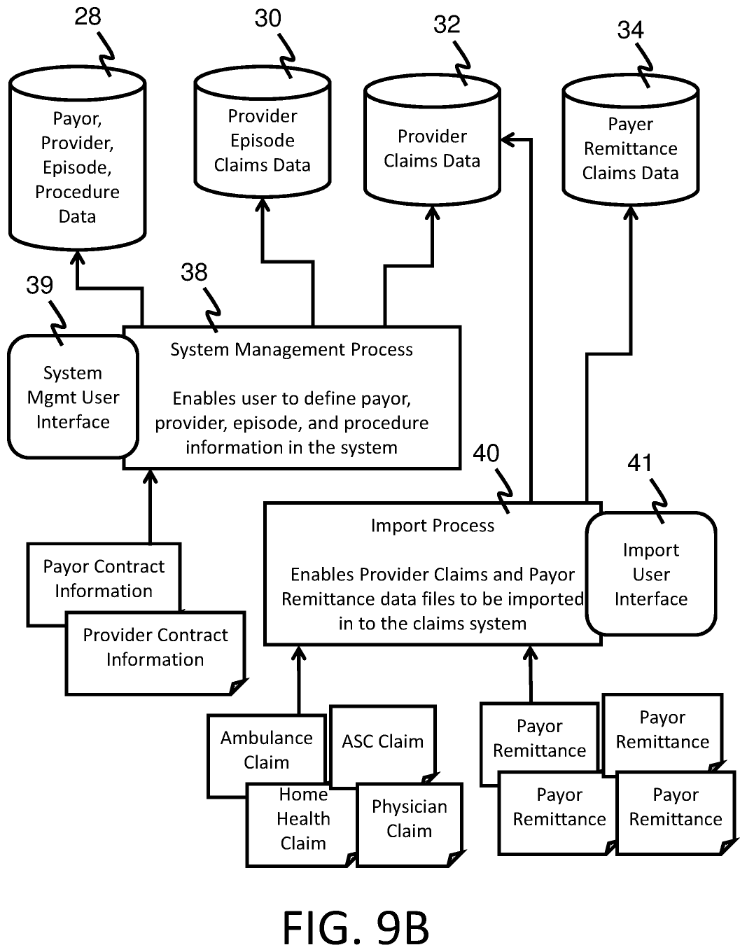

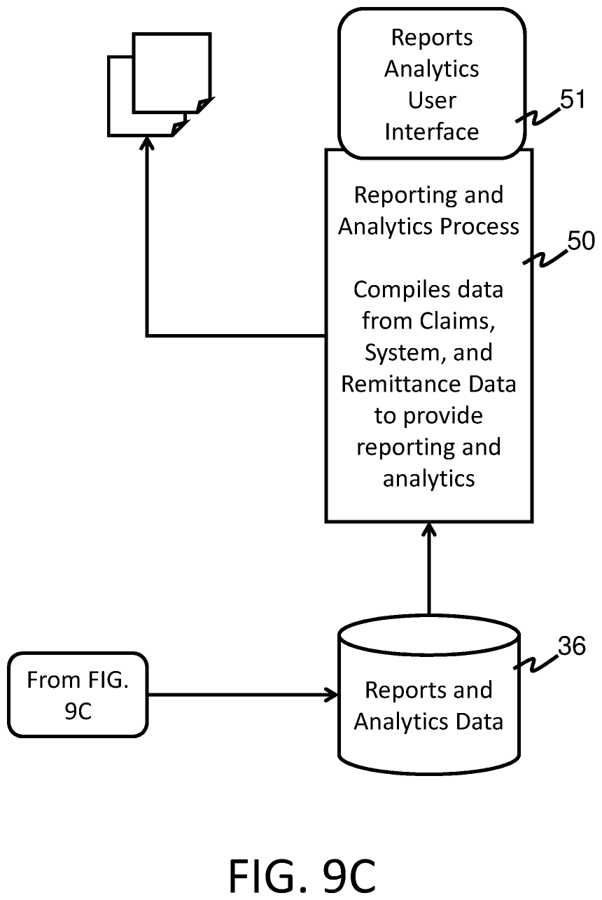

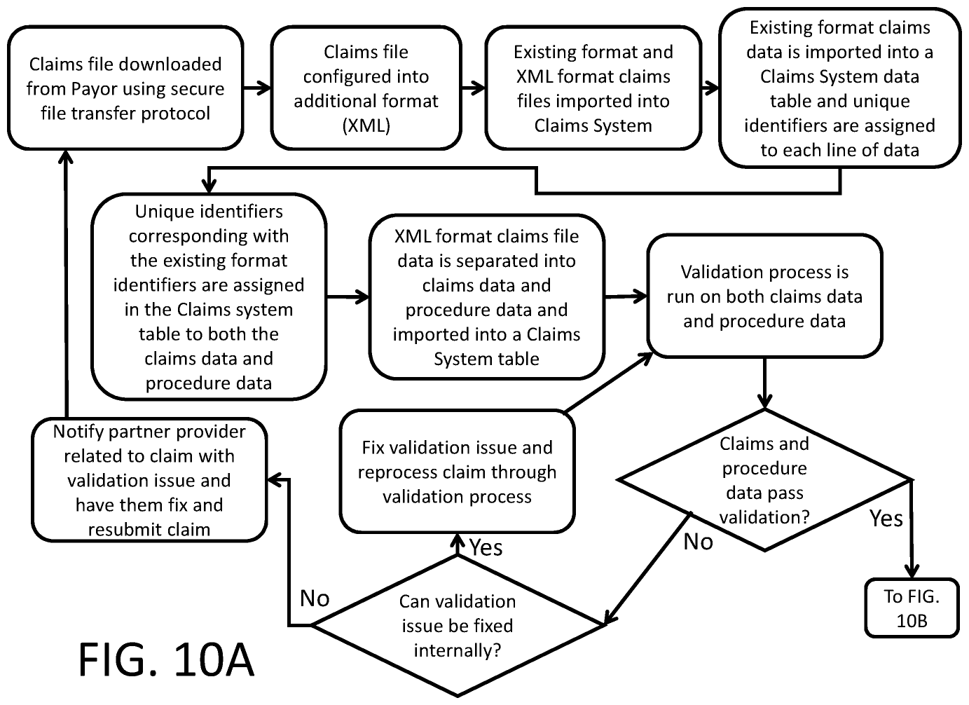

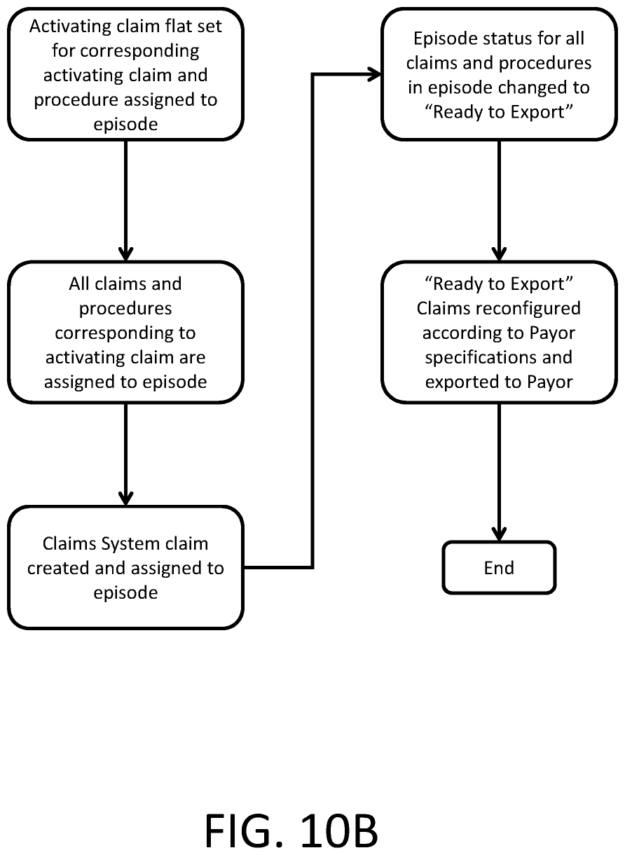

Claims system 10 (FIGS. 9-10) allows users to import, aggregate, retrieve, present, and reconfigure medical claims information from multiple data sources, as well as to export that information to multiple data sources. In one embodiment, the claims system 10 includes a payor, provider, episode, procedure data database 28 for storing payor, provider, episode, and procedure data; a provider episode claims data database 30 for storing provider episode claims data; a provider claims data database 32 for storing provider claims data; a payer remittance claims data database 34 for storing payer remittance claims data; and a reports and analytics data database 36 for storing reports and analytics data.

The system 10 may include a system management process 38 and user interface 39, an import process 40 and import user interface 41, an episode management process 42 and user interface 43, an export process 44 and user interface 45, a validation process 46 and validation user interface 47, a reconciliation process 48 and user interface 49, and a reporting and analytics process 50 and reports/analytics user interface 51. The system management process 38 and user interface 39 enable users to define payor, provider, episode, and procedure information in the claims system 10 and the import process 40 and user interface 41 enable provider claims and payor remittance data files to be imported into the claims system 10. The episode management process 42 manages the workflow and the assignment of provider claims data to episodes of care and the export process 44 and user interface 45 enable provider episode claims data to be reconfigured and exported to payors. Validation process 46 validates provider claims data, matches user defined parameters for information related to payor, provider, episode, and procedures and the reconciliation process 48, and matches provider claims data to remittance claims data. The reporting and analytics process 50 compiles data from claims, system, and remittance data to provide reporting and analytics.

The system management process 38 and the system management user interface 39 are in communication with one another and the payor, provider, episode, and procedure data database 28, the provider episode claims data database 30, and the provider claims data database 32. The import process 40 and import user interface 41 are in communication with one another and the provider claims data database 32 and the payor remittance claims data database 34. The episode management process 42 and episode management user interface 43 are in communication with one another and the provider episode claims data database 30 and the export process 44 and export user interface 45 are in communication with one another and the provider episode claims data database 30. Validation process 46 and validation user interface 47 are in communication with one another and the payor, provider, episode, procedure data database 28 and the provider claims data database 32. Reconciliation process 48 and reconciliation user interface 49 are in communication with one another and the payor, provider, episode, procedure data database 28, provider claims data database 32, and payor remittance claims data database 34. Reporting and analytics process 50 and reports/analytics user interface 51 are in communication with one another and the reports and analytics data database 36.

Payor contract information and provider contract information may be input into the claims system 10 using the system management user interface 39 and process 38. Ambulance claims, home health claims, ambulatory surgery center claims, physician claims and any other healthcare provider claims can be imported into the claims system 10 using the import process 40 and import user interface 41. Payor remittance data can be imported into the claims system 10 in a similar manner.

The System Management Process may include the following steps: 1) forming joint venture contracts with partner providers, e.g., specialists, surgeons, hospitals, anesthesiologists, physical therapists, and primary care physicians, and entering information regarding those contracts into the claims system 10; 2) forming contracts for episodes of care with partner providers and payors, e.g., Blue Cross Blue Shield; 3) entering Payor contract details into the claims system 10; 4) entering Partner provider contract details into the claims system 10; 5) entering episode of care information into the claims system 10 for each provider; 6) entering physician details into the claims system 10 for each contracted provider; 7) establishing provider associations with payors and physicians in the claims system 10; and 8) defining episodes of care in the claims system 10, which includes creating relationships between procedures, providers and payors for each episode of care.

The Import Process may include the following steps: 1) importing 837 claim files into the claim system 10; and 2) importing 837 XML files into the claims system 10. 837 claim files are generated by partner providers and are submitted to payors with a unique Joint Venture National Provider Identification (NPI) number. The payor or entity responsible for producing the 837 claim files advance routes the 837 claim files to a claims system 10 inbox (not shown). The “advanced routed” claims are then retrieved via a secure FTP (File Transfer Protocol) and imported into the claims system 10. The 837 files are then validated using the validation process described below.

The Validation Process for claims may include verifying the following: 1) that a claim has not previously been processed by comparing a ClaimID associated with the claim against all previously validated claims; 2) that a claim's payor information is in agreement with the assigned contract for that claim; 3) that a claim's provider information is in agreement with the assigned contract for that claim; 4) that a claim's physician information is in agreement with the assigned contract for that claim; 5) that a claim's procedure information is in agreement with the assigned contract for that claim; 6) that necessary procedure data is included in a claim; 7) that a claim identification number, place of service and diagnosis code are included in the claim; 8) that a claim's provider relationship information is in agreement with the assigned contract for that claim; 9) that a claim's procedure date is in agreement with the assigned contract date for that claim; 10) that all required subscribers data is included in a claim; 11) that all procedure dates are in agreement with timely filing dates for that claim; and 12) that all required patient data is included in a claim. Claims clearing the validation process are then moved to the Episode Management process.

The Episode Management Process may include the following steps: 1) determining episodes of care for payors; 2) linking all procedures within an episode of care to an activation procedure to create and begin an episode of care; 3) defining the day of service for an activating procedure which is used to calculate the warranty start and end periods associated with an episode of care; 4) verifying that all procedures occurred within the relevant warranty period; 5) assigning validated procedures to episodes of care; and 6) marking procedures assigned to episodes of care as ready for export.

The Export Process may include the following steps: 1) selecting claim type (institutional or professional), export type (encounter file or claims system file) payor, and joint venture to determine which files to export from claims system 10; 2) creating a system 837 claim file containing all procedures that are ready for export and chargeable claims for an episode of care; 3) entering an agreed upon fee for the episode of care into the claim amount field for the system 837 claim file and removing individual procedure amounts; 4) creating encounter 837 claim files containing all procedures that are ready for export and that do not contain chargeable claims for the episode of care; 5) reconfiguring encounter claim files to remove any chargeable information; and 6) sending all files meeting system and payor parameters to payors in 837 files for episode of care payment.

The Reconciliation Process may include the following steps: 1) payors receiving claims system 837 files and remitting payment information via an 835 file; 2) retrieving the 835 files via a secure FTP or other secure EDI (Electronic Data Interchange) method; 3) importing the 835 files into the claims system 10; 4) validating the 835 files by validating payor identifier, episode identifier, patient identifier, Joint Venture NPI, claimID, paid amount, and date of service from the imported 835 files with claims system data; and 5) reconciling payments when the payment amounts in the 835 files match the claim amounts in the system claims. Upon reconciliation, partner providers are remitted their agreed upon fee for services provided within episodes of care.

The Reporting and Analytics Process may include the following step: pulling data for providers, physicians, payors, episodes of care and facilities from the claims system 10 to create reports and analytics.

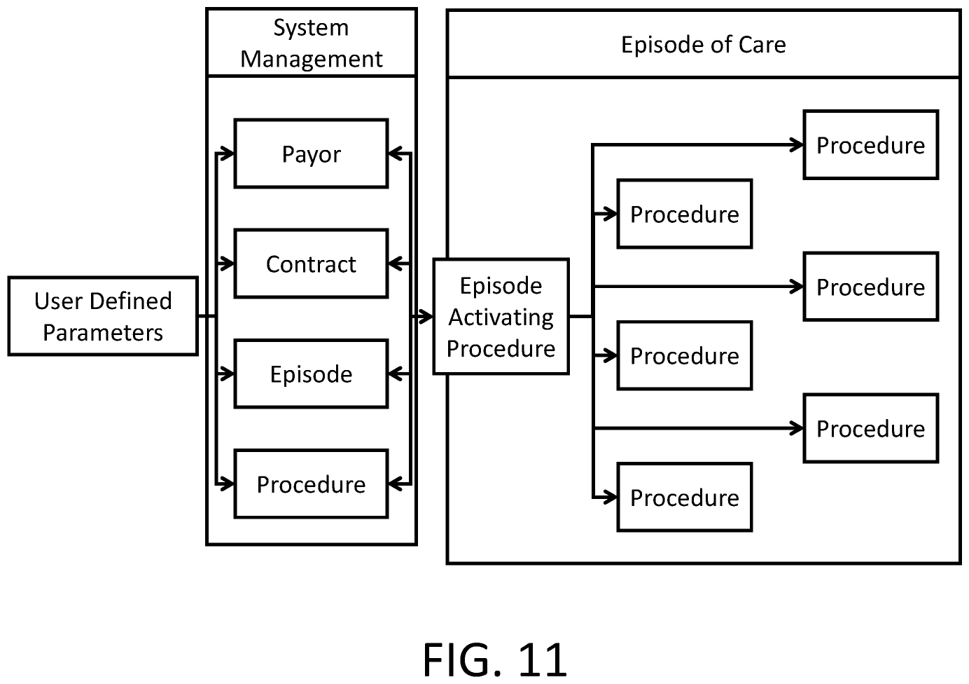

FIG. 11 is an Illustrative representation of how an episode of care is constructed in the claims system 10. The user defines the parameters for the payors, contracts, episodes, episode activating procedures and procedures within the system management section of the claims system 10. Each procedure and information related to the episode received from a partner provider is evaluated and matched to user defined parameters. Each procedure received is first screened for inclusion as an episode activating procedure. The episode activating procedure signifies the start of an episode of care and is the bridge between the user defined parameters and the other related procedures for episode assignment purposes. Once the episode activating procedure has been identified by the system, the claims system 10 is able to evaluate each corresponding procedure, matched them to the user defined parameters through claim file and system generated unique identifiers, and assign them to an episode of care.

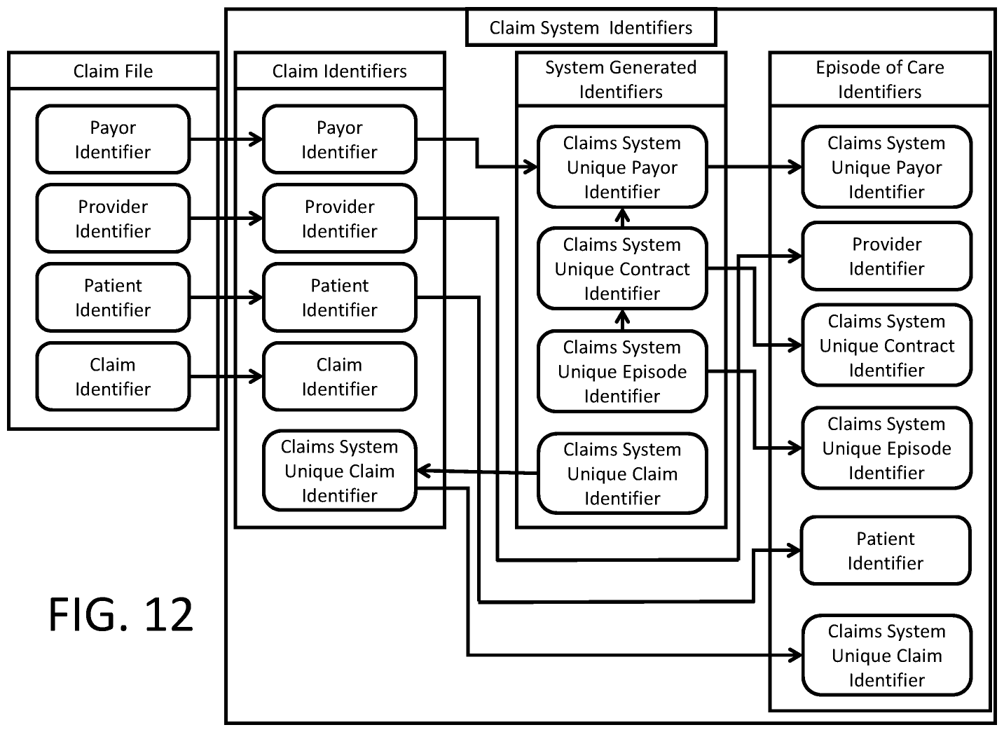

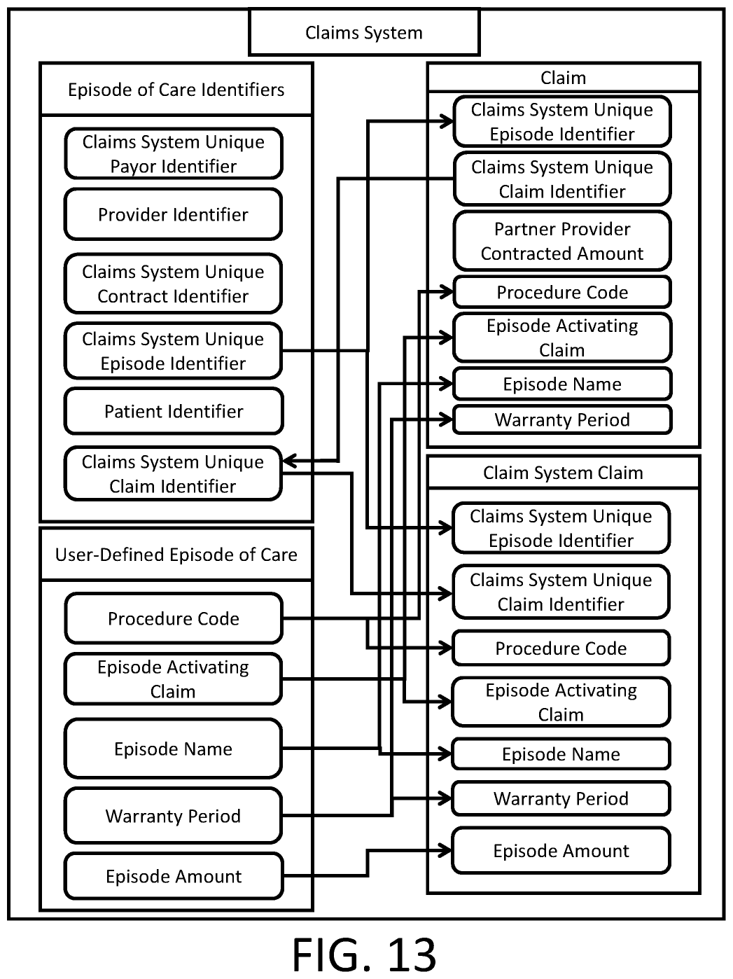

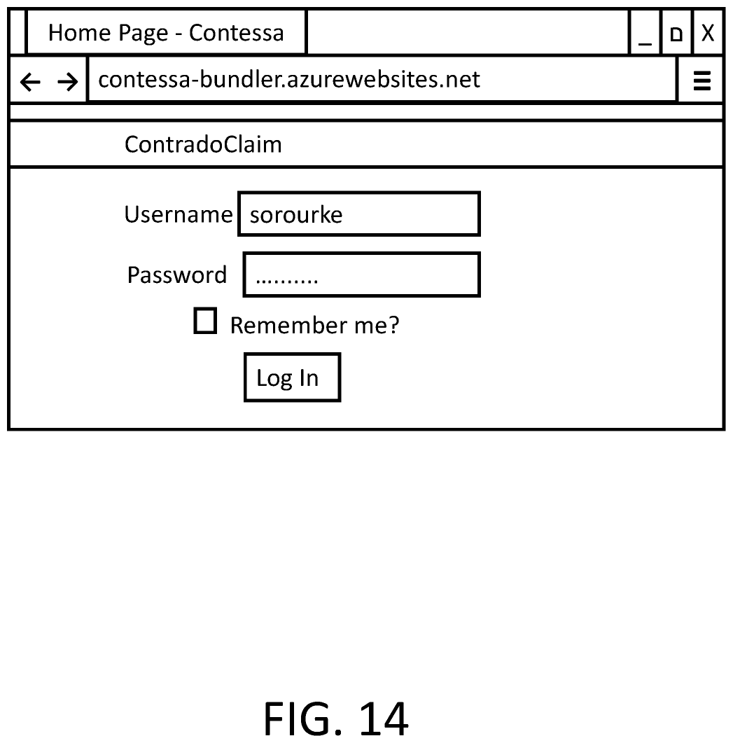

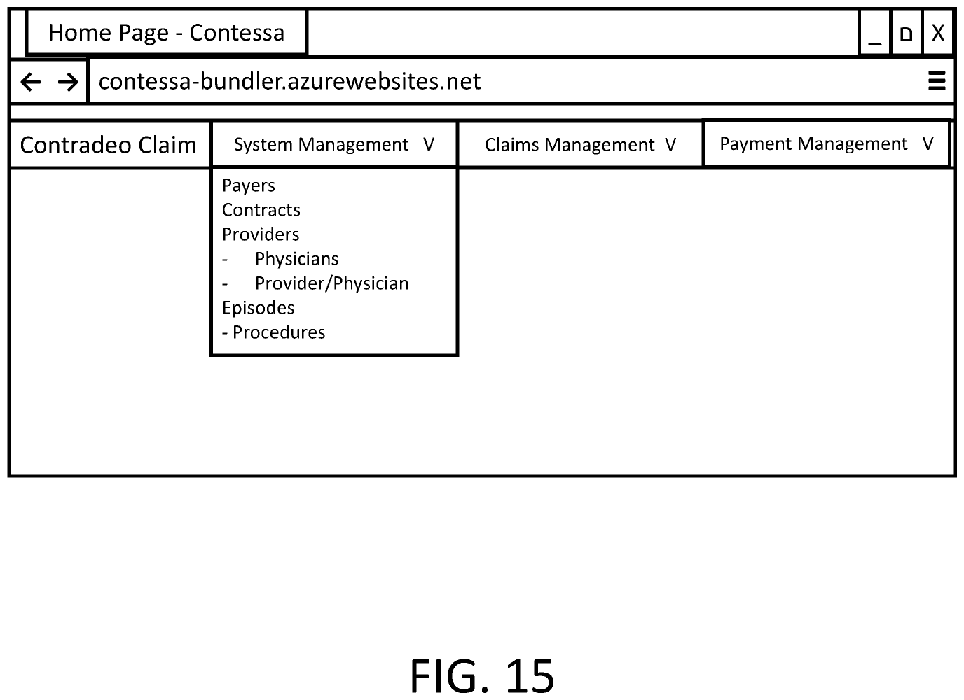

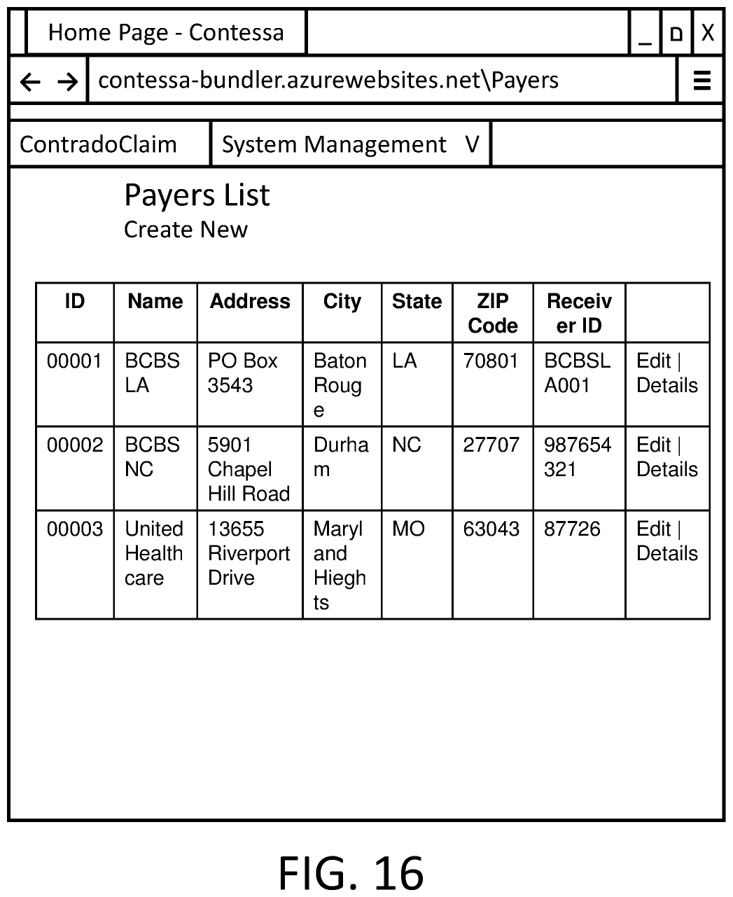

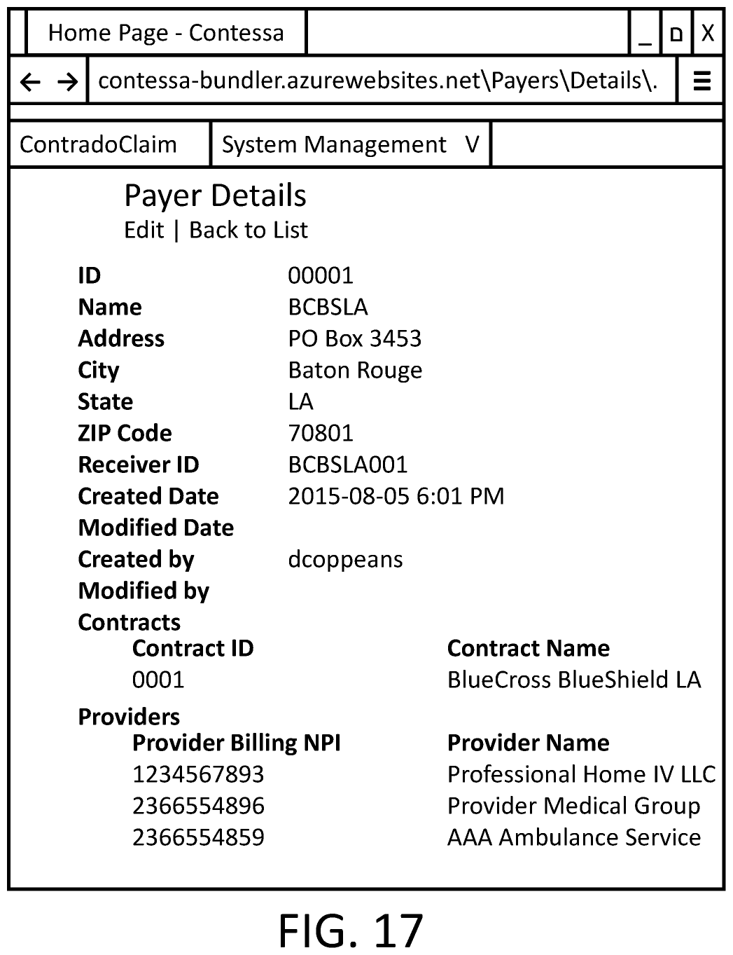

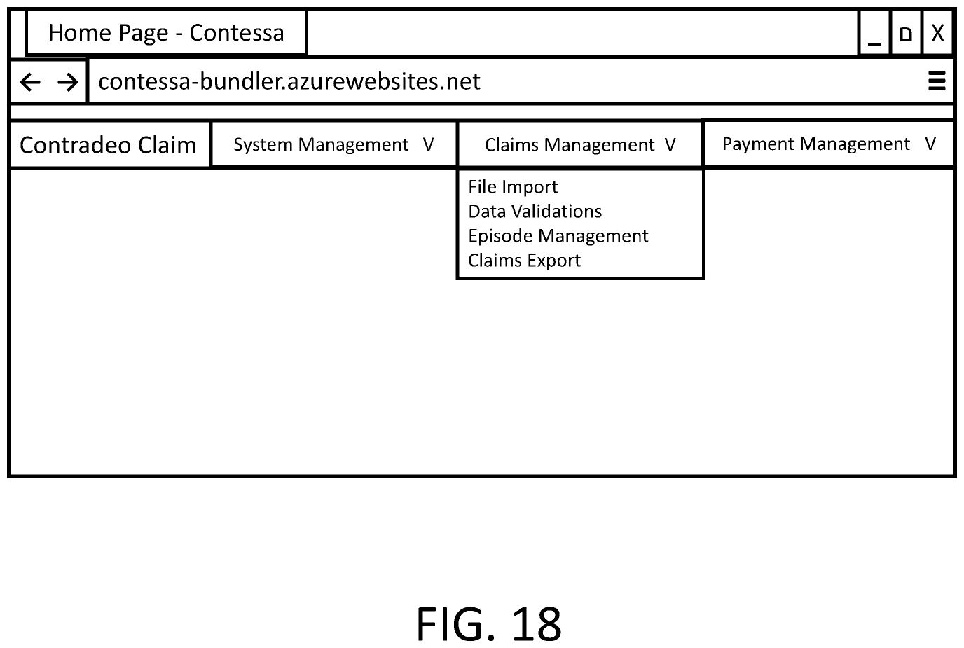

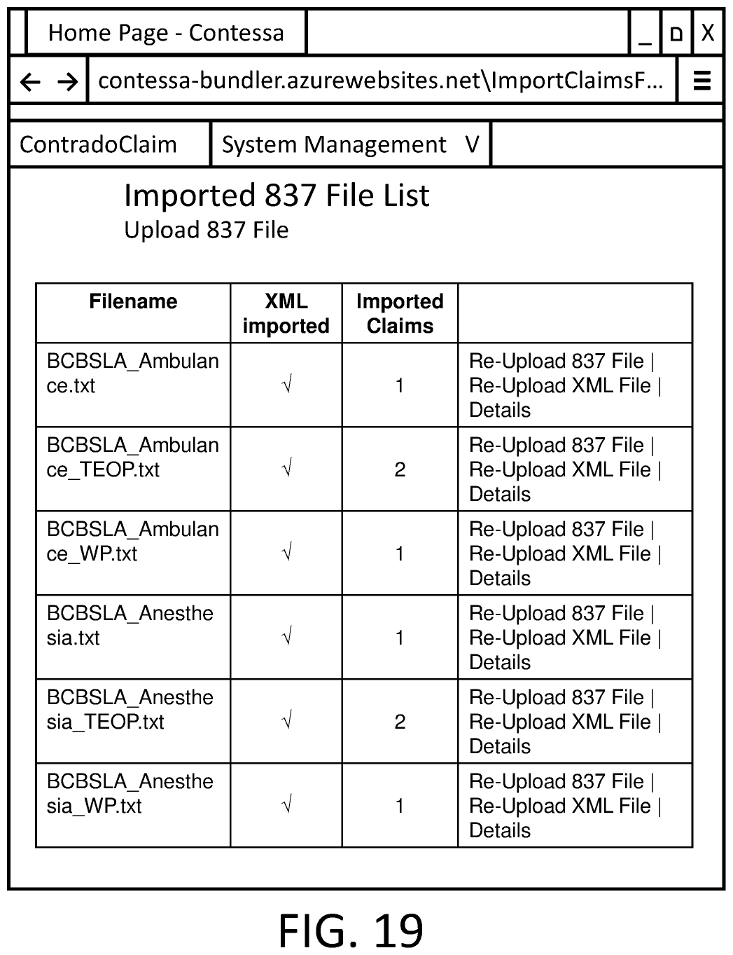

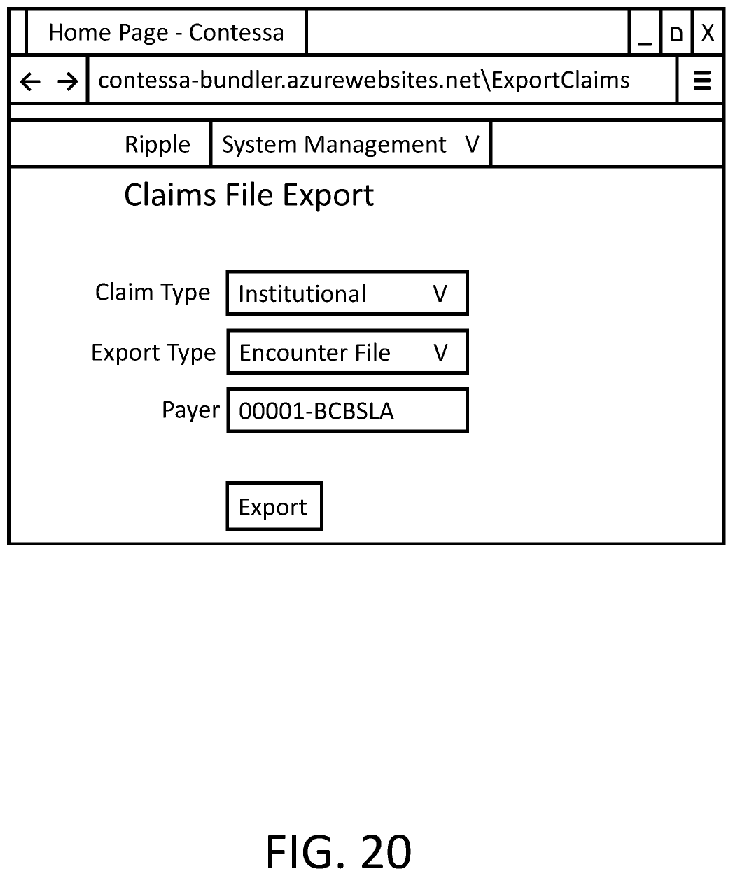

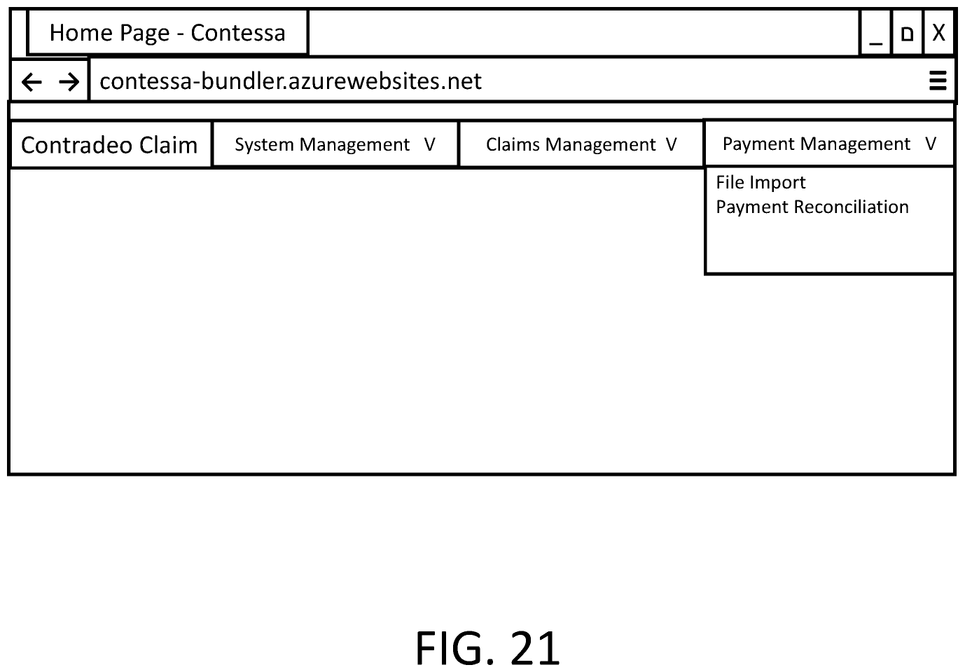

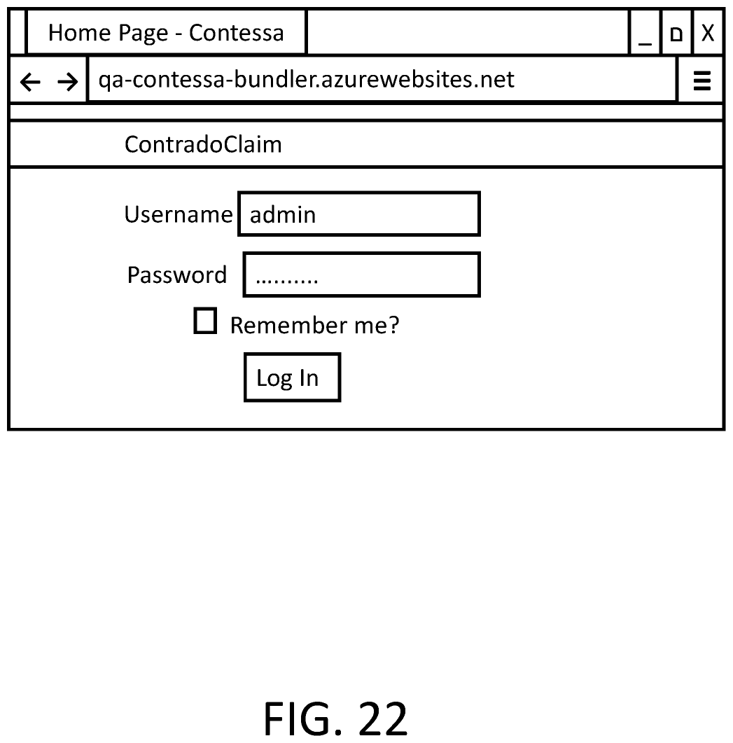

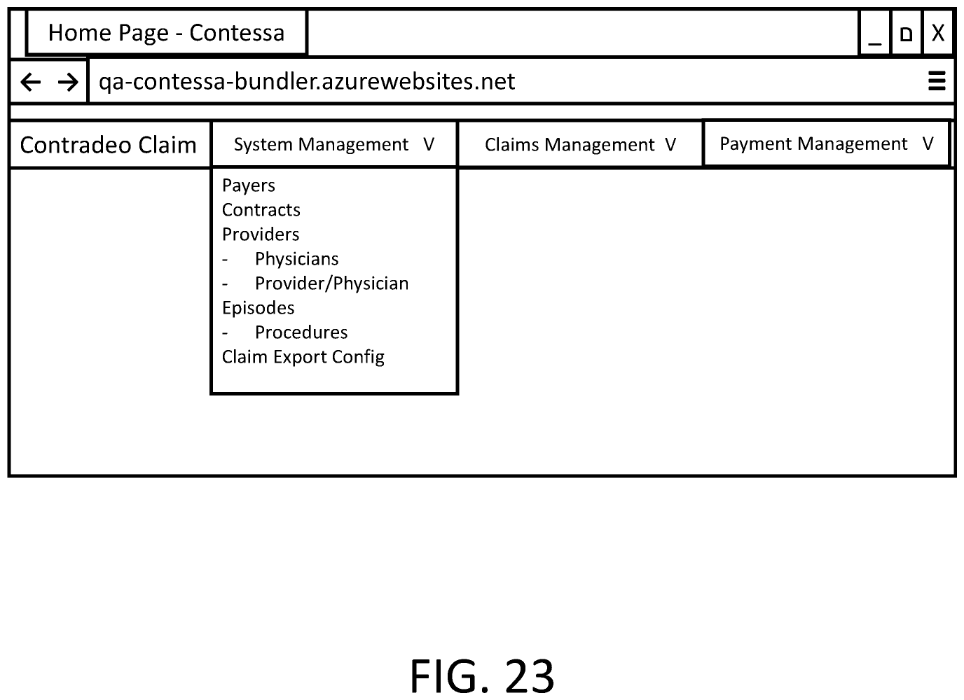

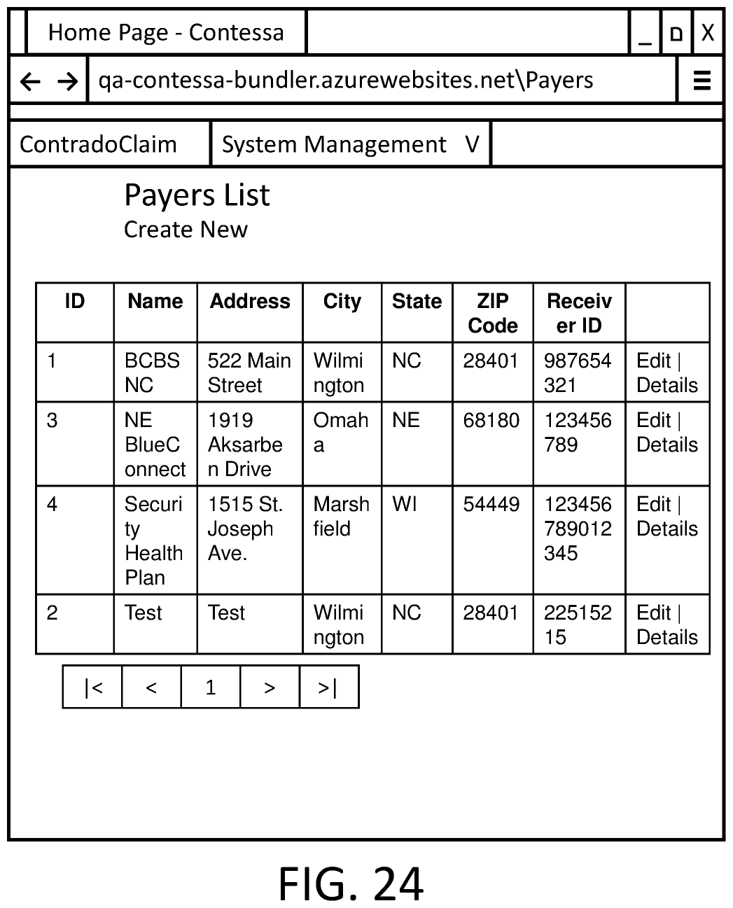

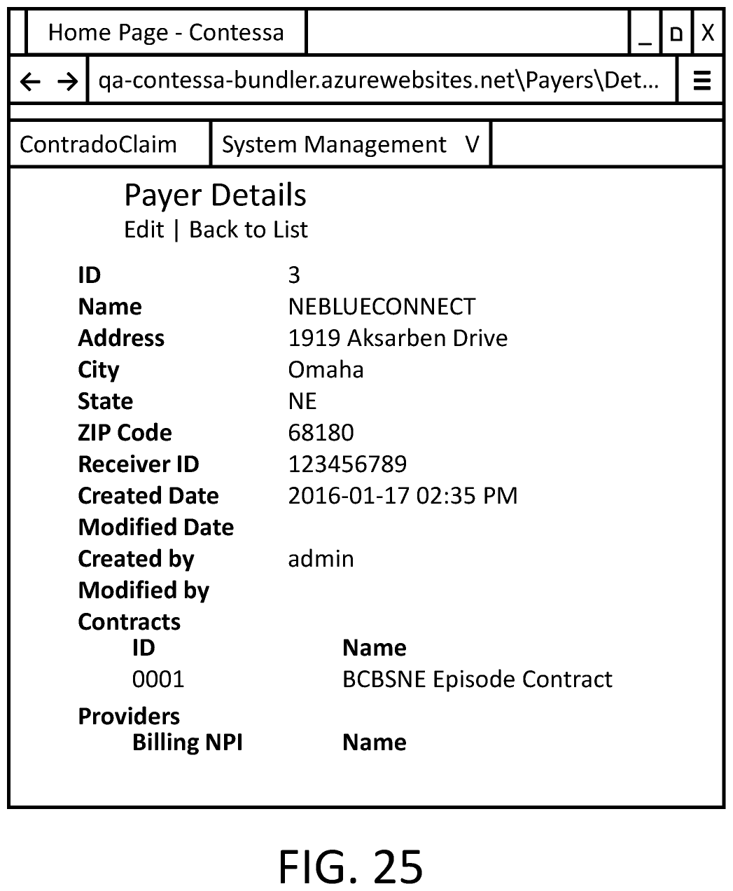

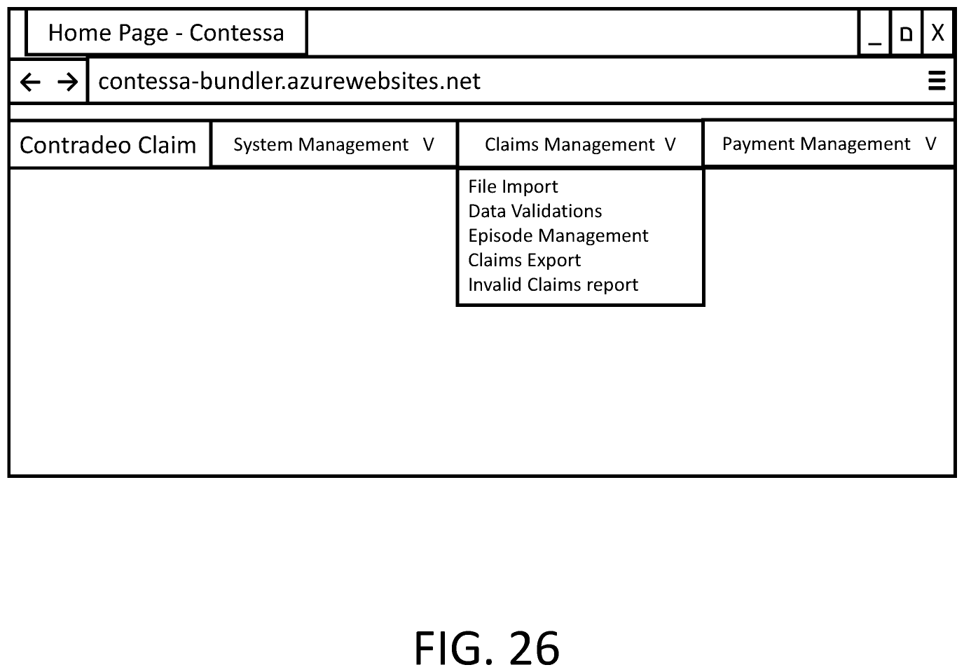

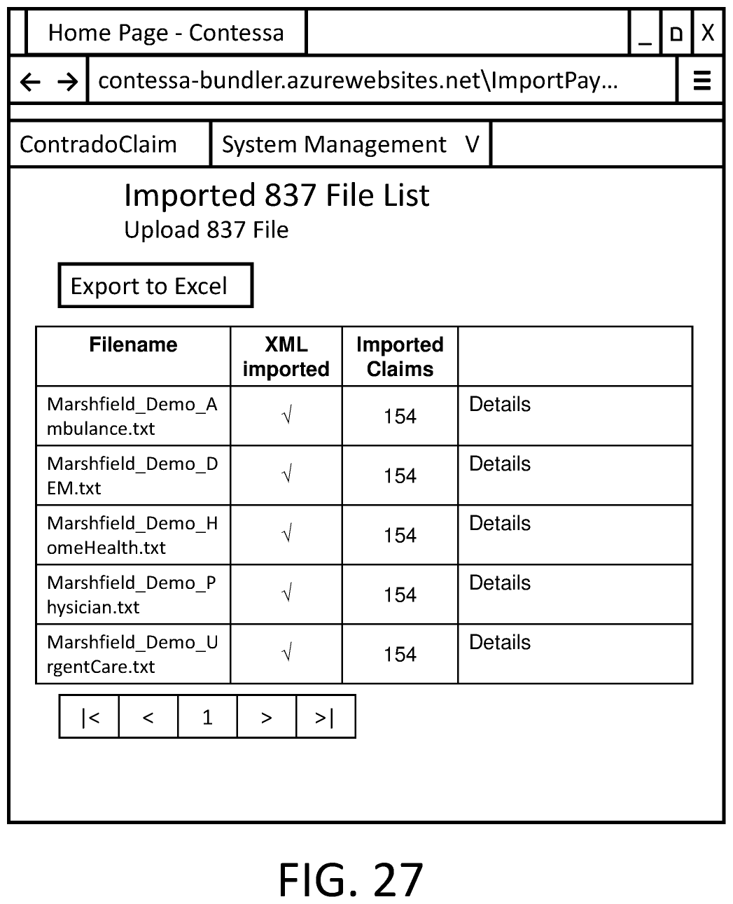

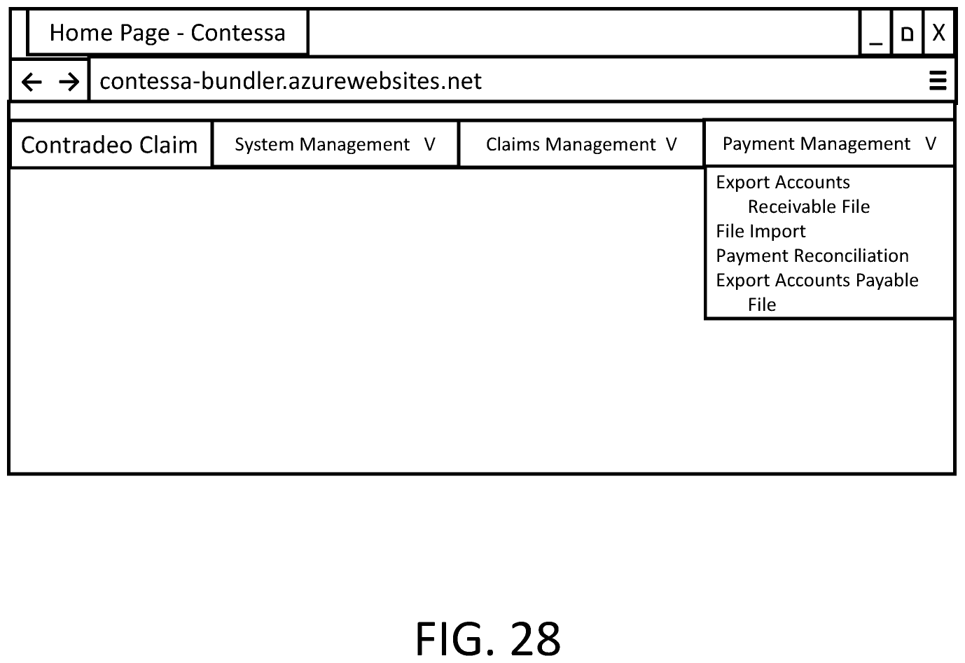

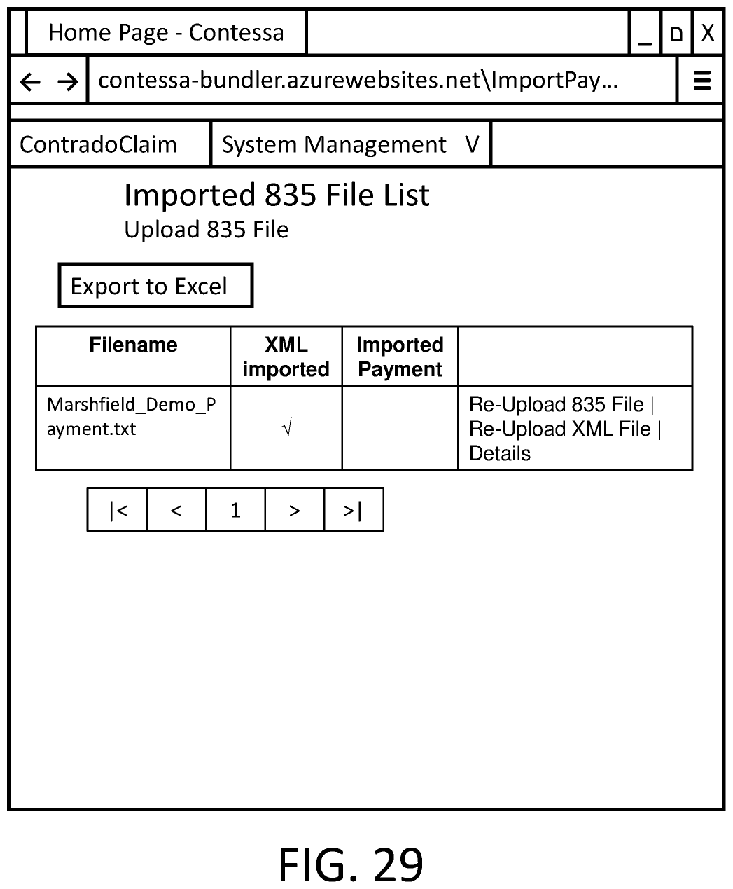

As shown in FIG. 12, each claim file received from partner providers contains identifiers to identify specific data elements in the claim file. Upon import of the claim file into the claims system 10, a unique system claim identifier is generated and linked to the claim identifier. When an episode of care is generated, the system 10 links both claim and system generated identifiers to the episode of care and they become identifiers of the episode of care. FIG. 13 illustrates this process in more detail.

FIG. 13 is an example of how the episode identification system works to identify and utilize relationships between the claims. In the first step, the Episode Activating claim is identified and the activating claim flag is set in the Claim using the User Defined Episode Activating claim information to identify the activating claim. Once the Episode Activating claim is identified, in step two the system uses the system Unique Identifier to identify all of the Episode of Care identifiers needed to assign all claims and procedures to the episode of care. In step three, using the identifier parameters from the second step, the system 10 screens all claims and procedures to compile a list of claims and procedures to be included in the episode of care. Next, a Unique system Episode identifier is assigned to each claim and procedure returned in the compiled list from the third step. An Episode name is assigned to each claim and procedure returned in the compiled list from the third step. A Warranty period is assigned to each claim and procedure returned in the compiled list from the third step. Once episode of care information is assigned to corresponding claims, a system claim is created and the corresponding Unique system claim identifier is assigned. Based on the Unique system claim identifier, the system episode identifier is assigned to the system claim. Based on unique system episode identifier, a procedure code is assigned to the system claim. Based on the unique system episode identifier, the episode name is assigned to the system claim. Based on the unique system episode identifier, the episode activating claim flag is assigned if necessary. Based on the unique system episode identifier, the warranty period is assigned to the system claim.

Thus, although there have been described particular embodiments of the present invention of a new and useful VALUE BASED HEALTH CARE CLAIMS PROCESSING SYSTEM, it is not intended that such references be construed as limitations upon the scope of this invention.

What is claimed is:

1. A system for generating a single health care claim for a payor, comprising:
a first database configured to store payor, provider, episode, and procedure data, wherein the payor, provider, episode, and procedure data includes
user defined parameters, and
payor contract information and provider contract information;
a second database configured to store episode of care data, wherein the episode of care data includes one or more episodes of care;
a third database configured to store provider claims data, wherein the provider claims data includes procedure information associated with a provider claim; and
at least one server configured to
receive, from a provider communication device, a provider claims file, wherein the provider claims file includes provider claims data,
validate a first group of one or more procedures of the provider claims data, wherein validating the first group of one or more procedures includes determining that each procedure of the first group of one or more procedures complies with the payor contract information and the provider contract information,
store the first group of one or more validated procedures in the third database,
convert a second group of one or more procedures stored in the third database into an episode of care, wherein converting the second group of one or more procedures into the episode of care includes
receiving, from the third database, a first procedure of the second group of one or more procedures, wherein
the first procedure is associated with a patient, and
the first procedure includes a procedure date, wherein the procedure date includes a date of service for the first procedure,
designating the first procedure as an activation procedure, wherein the activation procedure indicates a beginning of the episode of care,
receiving, from the third database, one or more second procedures of the second group of one or more procedures, wherein
each of the one or more second procedures are associated with the patient, and
each of the one or more second procedures include a procedure date,
identifying that the procedure date for each of the one or more second procedures occurred after the procedure date of the first procedure,
linking the one or more second procedures to the activation procedure,
assigning, based on the user-defined parameters, the one or more second procedures to the episode of care, and
generating, at the at least one server, a claims system episode identifier for the episode of care,
store the episode of care in the second database,
generate, in the at least one server, a claims system claim based on the episode of care, wherein generating the claims system claim includes
retrieving the episode of care from the second database,
including a fee amount in the claims system claim, and
assigning, to the claims system claim, the claims system episode identifier, and
sending, over a data network, the claims system claim to at least one payor.

2. The system of claim 1, wherein the provider claims data includes a plurality of provider claims, wherein each provider claim includes a payor identifier, a provider identifier, a patient identifier, and a claim identifier.

3. The system of claim 2, wherein the at least one server is further configured to assign, to each provider claim:
a claims system claim identifier;
a claims system payor identifier; and
a claims system contract identifier.

4. The system of claim 2, wherein the at least one server is further configured to assign, to each provider claim of the plurality of provider claims, the claims system episode identifier, a partner provider contracted amount, a procedure code, an episode-activating claim, an episode name, and a warranty period.

5. The system of claim 1, wherein generating, in the at least one server, the claims system claim based on the episode of care further includes assigning, to the claims system claim, a claims system claim identifier, a procedure code, an episode-activating claim, an episode name, and a warranty period.

6. The system of claim 1, wherein the payor, provider, episode, and procedure parameter data includes a procedure code, an episode-activating claim, an episode name, a warranty period, and the fee amount.

7. The system of claim 1, wherein the provider claims data comprises at least one of:
an ambulance claim;
a home health claim,
an ambulatory surgery center claim, or
a physician claim.

8. The system of claim 1, wherein the claims system episode identifier comprises a unique identifier among a plurality of claims system episode identifier.

9. The system of claim 1:
further comprising a fourth database configured to store payor remittance claims data; and
wherein the at least one server is further configured to
import a payor remittance data file into the system, and
store payor remittance claims data of the payor remittance file in the fourth database.

10. The system of claim 9:
further comprising a fifth database configured to store reports and analytics data; and
wherein the at least one server is further configured to
compile data from the first database, the second database, and the third database,
generate an analytics report based on the compiled data, and
store the generated analytics report in the fifth database.

11. A system for processing health care claims, comprising:
a first database configured to store payor, provider, episode, and procedure data, wherein the payor, provider, episode, and procedure data includes
user defined parameters, and
payor contract information and provider contract information, the payor contract information and provider contract information including warranty period data, episode-activating claim data, pricing data, provider data, episode data, and procedure data related to an episode of care, and provider contract information;
a second database configured to store episode of care data, wherein the episode of care data includes one or more episodes of care;
a third database configured to store provider claims data, wherein the provider claims data includes procedure information associated with a provider claim; and
at least one server configured to
receive, from a provider communication device, a provider claims file, wherein the provider claims file includes provider claims data,
validate a first group of one or more procedures of the provider claims data, wherein validating the first group of one or more procedures includes
determining that the provider claims data includes payor information that complies with the payor contract information,
determining that the provider claims data includes provider information that complies with the provider contract information,
determining that each procedure of the first group of one or more procedures complies with the payor contract information and the provider contract information, and
wherein the at least one server is configured to automatically send an invalidity notification to a provider that relates to the first group of one or more procedures in response to at least one of the payor information not complying with the payor contract information,
the provider information not complying with the provider contract information, or
at least a portion of the first group of one or more procedures not complying with the payor contract information and the provider contract information,
store the first group of one or more validated procedures in the third database,
convert a second group of one or more procedures stored in the third database into an episode of care, wherein converting the second group of one or more procedures into the episode of care includes
receiving, from the third database, a first procedure of the second group of one or more procedures, wherein
the first procedure is associated with a patient, and
the first procedure includes a procedure data, wherein the procedure date includes a data of service for the first procedure,
designating the first procedure as an activation procedure, wherein the activation procedure indicates a beginning of the episode of care,
calculating a beginning day of service for the activation procedure,
receiving, from the third database, one or more second procedures of the second group of one or more procedures, wherein
each of the one or more second procedures are associated with the patient, and
each of the one or more second procedures include a procedure date,
identifying that the procedure date of each of the one or more second procedures occurred after the procedure date of the first procedure,
linking the one or more second procedures to the activation procedure based on one or more system-generated unique identifiers of each of the one or more second procedures,
assigning, based on the user-defined parameters, the one or more second procedures to the episode of care, and
generating, at the at least one server, a claims system episode identifier for the episode of care,
store the episode of care in the second database,
generate, in the at least one server, a claims system claim based on the episode of care, wherein generating the claims system claim includes
retrieving, from the second database, the episode of care,
including a fee amount in the episode of care,
assigning, to the claims system claim, the claims system episode identifier, and
assigning to the claims system claim, the first procedure and the one or more second procedures,
formatting the claims system claim into a claims data file, and
sending, over a data network, the claims data file to at least one payor.

12. The system of claim 11:
further comprising a fourth database configured to store payor remittance claims data; and
wherein the at least one server is further configured to
import a payor remittance data file into the system, and
store payor remittance claims data of the payor remittance file in the fourth database.

13. The system of claim 12, wherein the at least one server is further configured to match at least a portion of the provider claims data to at least a portion of the remittance claims data.

14. The system of claim 13:
further comprising a fifth database configured to store reports and analytics data; and
wherein the at least one server is further configured
compile data from the first database, the second database, and the third database,
generate an analytics report based on the compiled data, and
store the generated analytics report in the fifth database.

15. The system of claim 12, wherein the remittance data file comprises an 835 remittance data file.

16. The system of claim 11, wherein the provider claims file comprises at least one of:
an 837 professional claims data file; or
an 837 institutional healthcare claims data file.

17. A computer-implemented method for generating a single health care claim for a payor, comprising:
receiving, at a server, a provider claims file from a provider communication device, the provider claims file including provider claims data;
retrieving, from a first database, payor, provider, episode, and procedure data, wherein the payor, provider, episode, and procedure data includes
user-defined parameters, and
payor contract information and provider contract information;
validating a first group of one or more procedures of the provider claims data, wherein validating the first group of one or more procedures includes determining that each procedure of the first group of one or more procedures complies with the payor contract information and the provider contract information, and in response to at least a portion of the first group of one or more procedures not complying with at least one of the payor contract information or the provider contract information, the at least one server is configured to automatically send an invalidity notification to a provider that relates to the at least a portion of the first group of the one or more procedures;
storing the first group of one or more procedures in a second database;
converting a second group of one or more procedures stored in the second database into an episode of care, wherein converting the second group one or more procedures into the episode of care includes
receiving, from the second database, a first procedure of the second group of one or more procedures, wherein
the first procedure is associated with a patient, and
the first procedure includes a procedure date, wherein the procedure date includes a date of service for the first procedure,
designating the first procedure as an activation procedure, wherein the activation procedure indicates a beginning of the episode of care,
receiving, from the second database, one or more second procedures of the second group of one or more procedures, wherein
each of the one or more second procedures are associated with the patient, and
each of the one or more second procedures includes a procedure date,
identifying that the procedure date for each of the one or more second procedures after the procedure date of the first procedure,
linking the one or more second procedures to the activation procedure,
assigning, based on the user-defined parameters, the one or more second procedures to the episode of care, and
generating, at the at least one server, a claims system episode identifier for the episode of care;
storing the episode of care in a third database;
generating, in the at least one server, a claims system claim based on the episode of care, wherein generating the claims system claim includes
retrieving, from the third database, the episode of care,
including a fee amount in the episode of care, and
assigning, to the claims system claim, the claims system episode identifier;
formatting the claims system claim into a claims data file; and
sending, over a data network, the claims data file to at least one payor.

18. The computer-implemented method of claim 18, further comprising:
receiving the payor data;
receiving the provider data;
receiving the episode data; and
generating the payor contract information and provider contract information, wherein generating the payor contract information and provider contract information includes
associating the provider data with the payor data, and
associating the procedure data with the provider data and the payor data based on the episode data.
